(19) United States
(12) Patent Application Publication
(10) Pub. No.: US 2025/0031961 A1
(43) Pub. Date: Jan. 30, 2025

(54) SYSTEMS AND METHODS FOR CONTROLLING DEVICE FUNCTIONALITY BASED ON DETERMINED REAL-WORLD ATTRIBUTES OF AN ENVIRONMENT

(71) Applicant: PLUME DESIGN, INC., Palo Alto, CA (US)

(72) Inventors: Miroslav SAMARDZIJA, Mountain View, CA (US); Shruti ROYYURU, Mountain View, CA (US); Liem Hieu Dinh VO, San Jose, CA (US)

(21) Appl. No.: 18/357,476

(22) Filed: Jul. 24, 2023

(51) Int. Cl. A61B 5/00

(52) U.S. Cl. CPC A61B 5/0004; A61B 5/411; A61B 5/6802; A61B 5/6826; A61B 5/742

(57) ABSTRACT

Disclosed are systems and methods that, via the disclosed functionality, can involve receiving (1) a request from a wearer of a wearable for the wearer to acquire an item and (2) from a secure storage device included in the wearable that securely maintains a set of restrictions associated with the wearer, the set of restrictions associated with the wearer. The systems and methods also include identifying at least one potential restriction associated with the item and determining, based on the set of restrictions associated with the wearer and the at least one potential restriction, a restriction associated with the item and the wearer. The systems and methods may also include executing at least one warning action in response to determining the restriction associated with the item and the wearer. Various other systems, methods, and computer-readable media are also disclosed.

### BRIEF DESCRIPTION OF THE DRAWINGS

The accompanying drawings illustrate a number of example embodiments and are a part of the specification. Together with the following description, these drawings demonstrate and explain various principles of the instant disclosure.

FIG. 1 is a block diagram of an example system for warning users of potential risks associated with potential purchases.

FIG. 2 is a block diagram of an example system that implements a system for warning users of potential risks associated with potential purchases.

FIG. 3 is a flow diagram of an example method for warning users of potential risks associated with potential purchases.

FIG. 4 shows a perspective view of an example smart ring device that may be used in connection with some of the systems and methods disclosed herein.

FIG. 5 shows a sequence diagram that illustrates operations that may be executed by components of one or more example systems disclosed herein during an example transaction.

FIG. 6 is a block diagram that illustrates receiving an additional potential restriction and transmitting the additional potential restriction to a wearable in accordance with some of the systems and methods disclosed herein.

Throughout the drawings, identical reference characters and descriptions indicate similar, but not necessarily identical, elements. While the example embodiments described herein are susceptible to various modifications and alternative forms, specific embodiments have been shown by way of example in the drawings and will be described in detail herein. However, the example embodiments described herein are not intended to be limited to the particular forms disclosed. Rather, the instant disclosure covers all modifications, equivalents, and alternatives falling within the scope of the appended claims.

### DETAILED DESCRIPTION OF EXAMPLE EMBODIMENTS

Wearable technology has undergone significant evolution in recent years, particularly in the domain of health and wellness. Wearable devices, ranging from fitness trackers and smartwatches to advanced medical wearables, may monitor various aspects of an individual's health and may provide valuable insights to enhance a wearer's wellbeing. By leveraging a host of sensors and software capabilities, such wearables can track a wide range of health metrics such as heart rate, blood oxygen levels, sleep patterns, activity levels, and even stress levels, all in real-time.

Furthermore, wearable technology is playing an increasingly pivotal role in personalized healthcare. These devices can be programmed to account for an individual's specific health conditions or lifestyle needs. For instance, they can monitor and alert users about variations in chronic conditions, remind them to take medication, and so forth. By integrating technology into everyday life, wearables in health and wellness provide a proactive approach to healthcare, facilitating early detection, prevention, and management of health issues.

Additionally, the integration of electronic payment capabilities within wearable technology has brought about a significant shift in the way transactions are conducted in today's digital age. Wearable devices may allow users to make seamless contactless payments, such as via suitably equipped Point-of-Sale (POS) terminals. This integration effectively transforms wearable devices into digital wallets, contributing to a cashless and card-less economy, and provides users with an effortless and secure way to manage their finances on-the-go.

Despite the considerable advancements in wearable technology for health monitoring and contactless payments, there exist certain challenges that limit their effectiveness and user experience. Traditional wearables typically function independently in health monitoring and transaction processing, lacking a system that bridges these functionalities to provide a comprehensive user-centric approach. While wearable devices can store health metrics and personal data, they lack the capability to apply this data during purchases. Similarly, the payment feature in wearable technology primarily focuses on the convenience of the transaction, but often overlooks the wearer's health considerations.

By way of example, a wearable device may be configured to store allergy data associated with a wearer. The stored allergy data may indicate that the wearer has an allergy to peanuts. The wearable device may also include wireless payment functionality. The user may employ this wireless payment functionality to purchase a candy bar that, unbeknownst to the wearer, contains peanuts. In this example, consuming the candy bar could place the wearer in significant danger of exposure to a potential allergen.

Consequently, there is a need for a more integrated and user-specific approach that leverages the combined potential of health monitoring and electronic payments in wearable technology to enhance both user safety and convenience.

The present disclosure is generally directed to systems and methods for warning users of potential risks associated with potential purchases. As will be described in more detail below, embodiments of the systems and methods disclosed herein may receive a request from a wearer of a wearable (e.g., a smart ring, a smart band, a smartphone, etc.) for the wearer to acquire (e.g., purchase, obtain, procure, etc.) an item. Embodiments may also receive, from a secure storage device included in the wearable that securely maintains a set of restrictions associated with the wearer (e.g., a set of allergies of the wearer), the set of restrictions associated with the wearer. Embodiments may also identify at least one potential restriction associated with the item (e.g., an allergen included as an ingredient in the item), and may determine, based on the set of restrictions associated with the wearer and the at least one potential restriction, a restriction associated with the item and the wearer (e.g., that the wearer has an allergy to the allergen included in the item). Embodiments may further execute at least one warning action in response to determining the restriction associated with the item and the wearer (e.g., present a warning to the user, a merchant, a third party to the transaction, etc.).

By seamlessly integrating personalized health monitoring and contactless payment functionalities, embodiments of the systems and methods disclosed herein may significantly enhance the utility and safety of wearable technology. The systems and methods disclosed herein may provide a unique wearer-centric approach, ensuring not only the convenience of making purchases but also the consideration of the wearer's health data during transactions. For instance, by warning a wearer attempting to purchase an item that could potentially cause an allergic reaction or conflict with dietary restrictions, embodiments of the systems and methods disclosed herein may aid in proactively safeguarding the wearer's health. This intelligent, real-time responsiveness may potentially reduce the risk of health complications related to purchases, leading to enhanced wearer safety.

Furthermore, the systems and methods disclosed herein may also promote awareness and encourage healthier purchasing habits. By integrating the wearer's health data into a purchase process, embodiments of the systems and methods disclosed herein may provide insights into how purchased items may impact the wearer's health. Such a feature could foster healthier lifestyle choices, contributing to the overall wellness of the wearer. Additionally, the ability to provide warnings regarding restrictions based on the wearer's personal health data may contribute to increased transparency during transactions, thus augmenting the wearer's control over their health management. This innovative integration of health data into the realm of electronic transactions may revolutionize the way wearable technology can be utilized in everyday life, driving a more personalized, safe, and informed user experience.

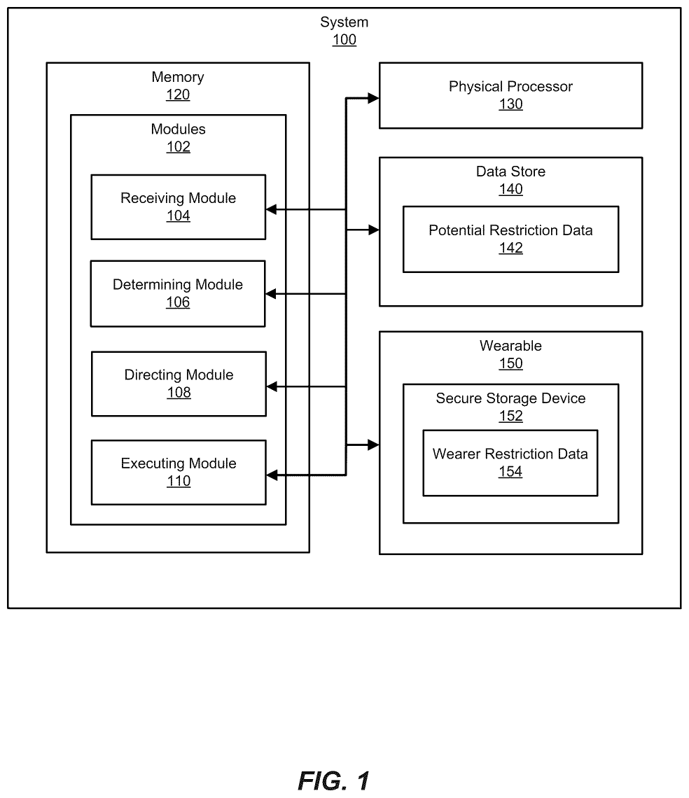

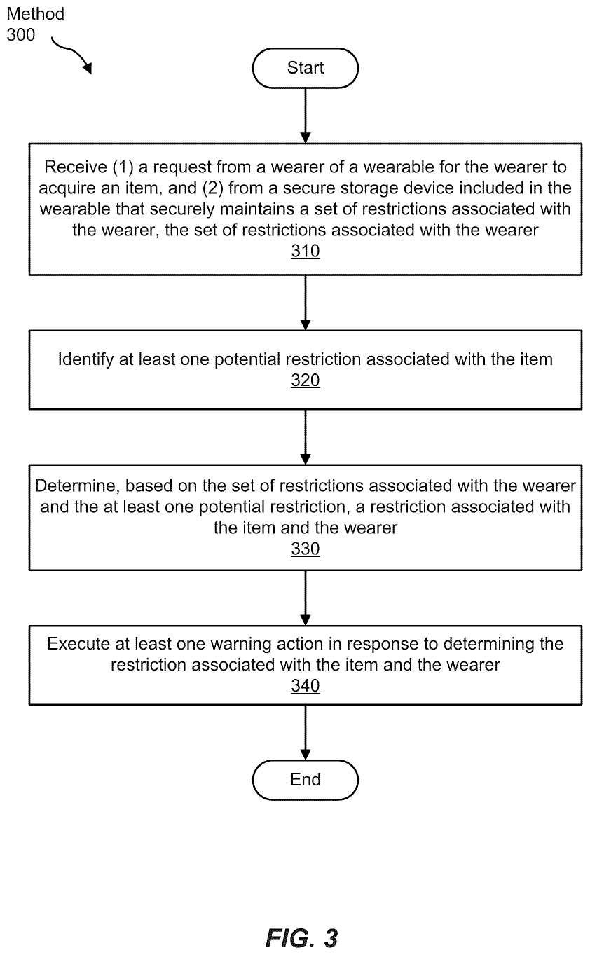

The following will provide, with reference to FIGS. 1-2 and 4-6, detailed descriptions of systems for warning users of potential risks associated with potential purchases. Detailed descriptions of corresponding computer-implemented methods will also be provided in connection with FIG. 3.

FIG. 1 is a block diagram of an example system 100 for warning users of potential risks associated with potential purchases. As illustrated in this figure, example system 100 may include one or more modules 102 for performing one or more tasks. As will be explained in greater detail below, modules 102 may include a receiving module 104 that may receive a request from a wearer of a wearable for the wearer to acquire an item. Receiving module 104 may also receive, from a secure storage device included in the wearable that securely maintains a set of restrictions associated with the wearer, the set of restrictions associated with the wearer.

Additionally, as shown in FIG. 1, example system 100 may also include an identifying module 106 that may identify at least one potential restriction associated with the item, and a determining module 108 that may determine, based on the set of restrictions associated with the wearer and the at least one potential restriction, a restriction associated with the item and the wearer.

Moreover, as further shown in FIG. 1, modules 102 may also include an executing module 110 that may execute at least one warning action in response to determining module 108 determining the restriction associated with the item and the wearer.

As further illustrated in FIG. 1, modules 102 may also include one or more memory devices, such as memory 120. Memory 120 generally represents any type or form of volatile or non-volatile storage device or medium capable of storing data and/or computer-readable instructions. In one example, memory 120 may store, load, and/or maintain one or more of modules 102. Examples of memory 120 include, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

As further illustrated in FIG. 1, example system 100 may also include one or more physical processors, such as physical processor 130. Physical processor 130 generally represents any type or form of hardware-implemented processing unit capable of interpreting and/or executing computer-readable instructions. In one example, physical processor 130 may access and/or modify one or more of modules 102 stored in memory 120. Additionally or alternatively, physical processor 130 may execute one or more of modules 102 to facilitate warning users of potential risks associated with potential purchases. Examples of physical processor 130 include, without limitation, microprocessors, microcontrollers, central processing units (CPUs), virtualized processors executed by physical processors, Field-Programmable Gate Arrays (FPGAs) that implement softcore processors, Application-Specific Integrated Circuits (ASICs), portions of one or more of the same, variations or combinations of one or more of the same, or any other suitable physical processor.

As also illustrated in FIG. 1, example system 100 may also include one or more stores of data, such as data store 140. Data store 140 may represent portions of a single data store or computing device or a plurality of data stores or computing devices. In some embodiments, data store 140 may be a logical container for data and may be implemented in various forms (e.g., a database, a file, file system, a data structure, and the like). Examples of data store 140 may include, without limitation, one or more files, file systems, data stores, databases, and/or database management systems such as an operational data store (ODS), a relational database, a NoSQL database, a NewSQL database, and/or any other suitable organized collection of data.

In at least one example, data store 140 may include potential restriction data 142 that may include data that may correlate items (e.g., items available for purchase) with potential restrictions. In some examples, potential restriction data 142 may include details about products or items that could pose potential risks or are unsuitable for certain individuals under some circumstances. In some examples, potential restriction data 142 may include a structured data set of attributes (e.g., ingredients, aspects, ownership identifiers, etc.) associated with items. For example, in a health context, potential restriction data 142 could link specific food items, like peanuts or shellfish, with potential allergic reactions for those with relevant allergies. Similarly, in a medication context, this data could associate certain over-the-counter drugs with potential harmful interactions for individuals on particular prescription medications.

As will be described in additional detail below, when a wearer attempts to purchase an item, one or more of the systems disclosed herein may access and/or analyze potential restriction data 142 in conjunction with the wearer's personalized restriction data (e.g., allergies, health conditions, etc.). This may allow one or more of the systems disclosed herein to determine if there is a risk or restriction relevant to the user for the specific item they are trying to acquire, ensuring a personalized, health-conscious shopping experience.

As is further shown in FIG. 1, example system 100 may also include a wearable 150. In some examples, a “wearable” or “wearable device” generally includes any device and/or devices designed and/or intended to be worn by a wearer and/or integrated into clothing. These devices may have the ability to connect to the internet, sync with other devices (e.g., mobile phones, personal computers, tablet computers, and the like) and provide a variety of features including, but not limited to, tracking physical activity (e.g., steps, heart rate, calories burned, and the like), monitoring biometric information (e.g., blood pressure, blood glucose levels, sleep quality, and the like), providing notifications (e.g., voice calls, emails, text messages, reminders, and the like), supporting navigation (e.g., via positioning systems, Wi-Fi, triangulation, and the like), making contactless payments, voice control, and/or gesture recognition.

Smart rings are a specific type of wearable technology that may be worn on a wearer's finger, similar to a traditional ring. They can be designed to provide various functionalities like those mentioned above and are often focused on discrete or minimalist design to maintain an outward style aspect of a ring while adding smart capabilities. Some may even include bio-sensing features such as measuring stress, body temperature, or providing an electrocardiogram (ECG). These features can vary greatly depending on the particular make and model of the smart ring, and hence this disclosure is not limited to any particular wearable device.

In additional or alternative examples, a “wearable” may include any device capable of (1) securely maintaining a set of restrictions associated with a wearer, and (2) transmitting that data to one or more of modules 102 (e.g., receiving module 104), such as a smart ring, a smart band, a smartphone, and so forth.

As further shown in FIG. 1, wearable 150 may include a secure storage device 152 that may securely store wearer restriction data 154. Secure storage device 152 may include any component within wearable 150 that is designed to securely store sensitive data. Given the nature of data it handles, secure storage device 152 may employ various security measures such as encryption and access control mechanisms to ensure the confidentiality, integrity, and availability of the data stored therein.

Wearer restriction data 154 may include any information securely stored within secure storage device 152 that is associated with the wearer of wearable 150. Wearer restriction data 154 may include information regarding a wearer's specific health conditions, allergies, lifestyle needs, legal restrictions, or other factors that might restrict or impact their ability to safely purchase, consume, and/or interact with certain items, products, and/or ingredients therein. In some examples, and without limitation, wearer restriction data 154 may include data relating to one or more of the following:

Health Conditions: Information about specific health conditions of the wearer, such as diabetes, hypertension, cardiovascular disease, or epilepsy, which may affect their ability to safely consume or use certain products.

Allergy Information: Data regarding allergies that the wearer may have. This could range from food allergies (like peanuts, shellfish, or gluten) to material allergies (like latex or certain metals), which could restrict the type of products they can safely interact with.

Lifestyle Needs: Information pertaining to the wearer's lifestyle choices, such as being vegan or vegetarian, following a Kosher or Halal diet, or preferring organic products, which may restrict the type of items they wish to purchase or consume.

Legal Restrictions: Information about any legal restrictions the wearer may be under. For example, age restrictions (being under the legal drinking or smoking age), probation conditions, or restraining orders, which could impact the wearer's ability to purchase or interact with certain items.

Other Factors: Information about any other factors that might restrict or impact the wearer's ability to safely purchase, consume, and/or interact with certain items. This could include data about their fitness level, physical limitations, or specific dietary requirements (like low sodium, low sugar, or high protein needs).

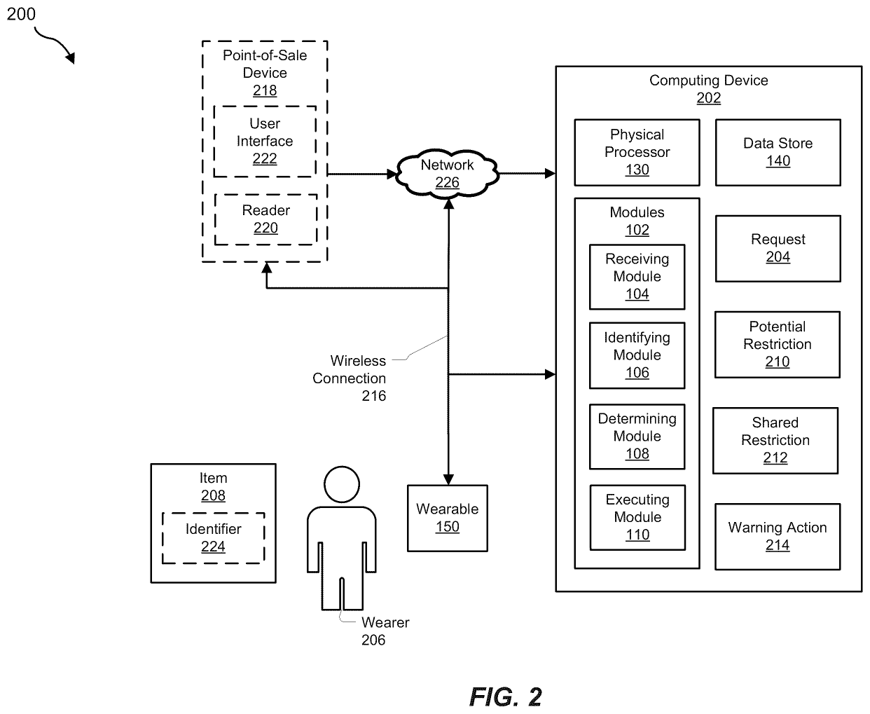

Example system 100 in FIG. 1 may be implemented in a variety of ways. For example, all or a portion of example system 100 may represent portions of an example system 200 (“system 200”) in FIG. 2. FIG. 2 is a block diagram of an example system 200 that implements a system for warning users of potential risks associated with potential purchases. As shown in FIG. 2, system 200 may include a computing device 202 in communication with wearable 150. In at least one example, computing device 202 may be programmed with one or more of modules 102.

In at least one embodiment, one or more of modules 102 from FIG. 1 may, when executed by computing device 202, computing device 202 to perform one or more operations to warn users of potential risks associated with potential purchases. For example, as will be described in greater detail below, receiving module 104 may cause computing device 202 to receive a request (e.g., request 204) from a wearer (e.g., wearer 206) of a wearable (e.g., wearable 150) for the wearer to acquire an item (e.g., item 208). Additionally, receiving module 104 may also cause computing device to, receive, from a secure storage device included in the wearable (e.g., secure storage device 152) that securely maintains a set of restrictions associated with the wearer (e.g., wearer restriction data 154), the set of restrictions associated with the wearer.

Additionally, identifying module 106 may cause computing device 202 to identify at least one potential restriction (e.g., potential restriction 210) associated with the item, and determining module 108 may cause computing device 202 to determine, based on the set of restrictions associated with the wearer and the at least one potential restriction, a restriction associated with the item and the wearer (shared restriction 212). Moreover, executing module 110 may cause computing device 202 to execute at least one warning action (e.g., warning action 214) in response to determining the restriction associated with the item and the wearer.

Computing device 202 generally represents any type or form of computing device capable of reading and/or executing computer-executable instructions. Examples of computing device 202 include, without limitation, servers, desktops, laptops, tablets, cellular phones, (e.g., smartphones), personal digital assistants (PDAs), multimedia players, embedded systems, wearable devices (e.g., smart watches, smart glasses, and the like), gaming consoles, combinations of one or more of the same, or any other suitable mobile computing device.

In some examples, wearable 150 may communicate with one or more components included in computing device 202 (e.g., one or more of modules 102) via a wireless connection 216. Wireless connection 216 may allow wearable 150 and computing device 202 to exchange information wirelessly, facilitating the execution of various operations to warn users of potential risks associated with potential purchases. Wireless connection 216 may be based on one or several wireless technologies or protocols, including but not limited to Wi-Fi, a Bluetooth wireless communication protocol, cellular networks, or a Near-Field Communication (NFC) wireless communication protocol. Wireless connection 216 may enable transfer of data, such as the wearer's restriction data from the secure storage device in the wearable, to the computing device (e.g., via receiving module 104). Similarly, wireless connection 216 may enable transmission of warning actions or notifications from computing device 202 back to wearable 150. In some examples, as will be described in greater detail below, wireless connection 216 may further enable wearable 150 to connect to and/or communicate with one or more devices via network 226.

As shown in FIG. 2, in some examples, wearable 150 may also be capable of communicating (e.g., via wireless connection 216) with a point-of-sale device such as point-of-sale device 218. Point-of-sale device 218 may include or represent a device used to complete retail transactions. Point-of-sale device 218 may be capable of accepting electronic payments and/or could be located at merchant locations. It may include a cash register, dedicated terminal, an integrated system equipped with software that handles transactions and processes payments, and so forth.

Wearable 150 may be capable of communicating with point-of-sale device 218, potentially via wireless connection 216. This interaction may facilitate an electronic payment process where the wearable device can act as a digital wallet. Notably, point-of-sale device 218 may also be able to transmit item-related information to wearable 150 and/or computing device 202, enabling real-time assessment of potential risks associated with the item being purchased, based on the wearer's restriction data.

As further shown in FIG. 2, in some examples, point-of-sale device 218 may include a reader 220 and/or a user interface 222. Reader 220 may include any device that may read (e.g., scan, analyze, interpret, decode, etc.) an identifier corresponding to an item, such as identifier 224 associated with item 208. In some examples, reader 220 may include an optical reader device capable of reading an optical identifier, and identifier 224 may include an optical identifier that may be read by reader 220.

Reader 220 may include, without limitation, an optical reader device or a barcode scanner that can read visual identifiers like barcodes or QR codes. Additionally or alternatively, reader 220 may also include an RFID reader or a magnetic strip reader, depending on the type of identifiers used. In some examples, reader 220 may capture and translate identifier data (e.g., read from identifier 224) into a format that the system can use to identify an item (e.g., item 208) and retrieve relevant information about it, such as its price or potential restrictions.

Identifier 224 may include a unique marker or tag associated with item 208 that can be read by reader 220. Identifier 224 may represent specific information about the item in a coded format, such as, without limitation, an optical identifier like a barcode or QR code, a RFID tag, a magnetic strip, and so forth. Identifier 224 may enable example system 200 to uniquely identify item 208 and fetch relevant data about it from a suitable storage location (e.g., potential restriction data 142 included in data store 140), which could include product details, pricing information, and potential restriction data.

User interface 222 may include any device or functionality that allows interaction between the system (e.g., e.g., example system 100 and/or example system 200) and a user (e.g., wearer 206, a merchant, etc.). Although shown as part of reader 220, user interface 222 may additionally or alternatively be included as part of computing device 202 and/or wearable 150. User interface 222 may include any component or set of components that may enable the user to input data, receive feedback, or control the operations of the system. For instance, user interface 222 could include a touchscreen for input, a display for showing item details or transaction information, buttons for controlling certain functions, and so forth. In some contexts, user interface 222 could also include components for presenting warning notifications or alerts to a user (e.g., wearer 206, a merchant, etc.), based on analysis of potential restriction data 142 and/or wearer restriction data 154.

As also shown in FIG. 2, in some examples, example system 200 may also include a network 226. Network 226 generally represents any medium or architecture capable of facilitating communication and/or data transfer between computing device 202 and one or more other network-enabled devices. For example, the network 226 can be, but is not limited to, a Wi-Fi network, a local area network (LAN), a wide-area network (WAN), and/or any other type of network that can facilitate connectivity among devices at a location and/or with a cloud service (e.g., Bluetooth™, and the like). Network 226 may facilitate communication or data transfer using wireless or wired connections. Examples of network 226 include, without limitation, an intranet, a WAN, a LAN, a Personal Area Network (PAN), the Internet, Power Line Communications (PLC), a cellular network (e.g., a Global System for Mobile Communications (GSM) network, a code-division multiple access (CDMA) network, a Long-Term Evolution (LTE) network, a Fifth-Generation (5G) network, and the like), universal serial bus (USB) connections, and the like. In some embodiments, network 226 may facilitate communication between computing device 202, wearable 150, and/or point-of-sale device 218.

In some examples, network 226 may include not just local physical networks but also software-defined networks (SDNs), virtual private networks (VPNs), or any architecture capable of facilitating communication and data transfer across geographically and/or logically dispersed locations. In some cases, network 226 may not be restricted to a single physically restricted network but can be a collection of interconnected networks that operate as a single entity by virtue of software control or through virtual connections. This arrangement can enable the user to cause a warning action to be taken in a physically remote location, provided the devices are part of the same software-defined or virtual network.

Furthermore, the control of connected devices may not be limited to a single network. In some examples, warning actions may result in a message being sent to another network where the corresponding action is taken based on the message received. In this way, network 226 represents any medium or architecture capable of facilitating communication, data transfer, or remote management of connected devices, across various physical or virtual networks.

In at least one example, computing device 202 may be a computing device programmed with one or more of modules 102. All or a portion of the functionality of modules 102 may be performed by computing device 202 and/or any other suitable computing system. As will be described in greater detail below, one or more of modules 102 from FIG. 1 may, when executed by at least one processor of computing device 202, may enable computing device 202 to provide personalized home automation.

Many other devices or subsystems may be connected to example system 100 in FIG. 1 and/or example system 200 in FIG. 2. Conversely, all of the components and devices illustrated in FIG. 1 and FIG. 2 need not be present to practice the embodiments described and/or illustrated herein. The devices and subsystems referenced above may also be interconnected in different ways from those shown in FIG. 2. Example system 100 and example system 200 may also employ any number of software, firmware, and/or hardware configurations. For example, one or more of the example embodiments disclosed herein may be encoded as a computer program (also referred to as computer software, software applications, computer-readable instructions, and/or computer control logic) on a computer-readable medium.

FIG. 3 is a flow diagram of an example method 300 for warning users of potential risks associated with potential purchases. The steps shown in FIG. 3 may be performed by any suitable computer-executable code and/or computing system, including example system 100 in FIG. 1, example system 200 in FIG. 2, and/or variations or combinations of one or more of the same. In one example, each of the steps shown in FIG. 3 may represent an algorithm whose structure includes and/or is represented by multiple sub-steps, examples of which will be provided in greater detail below.

As illustrated in FIG. 3, at step 310, one or more of the systems described herein may receive a request from a wearer of a wearable for the wearer to acquire an item. For example, receiving module 104 may, as part of computing device 202 in FIG. 2, cause computing device 202 to receive request 204 from wearer 206 for wearer 206 to acquire item 208.

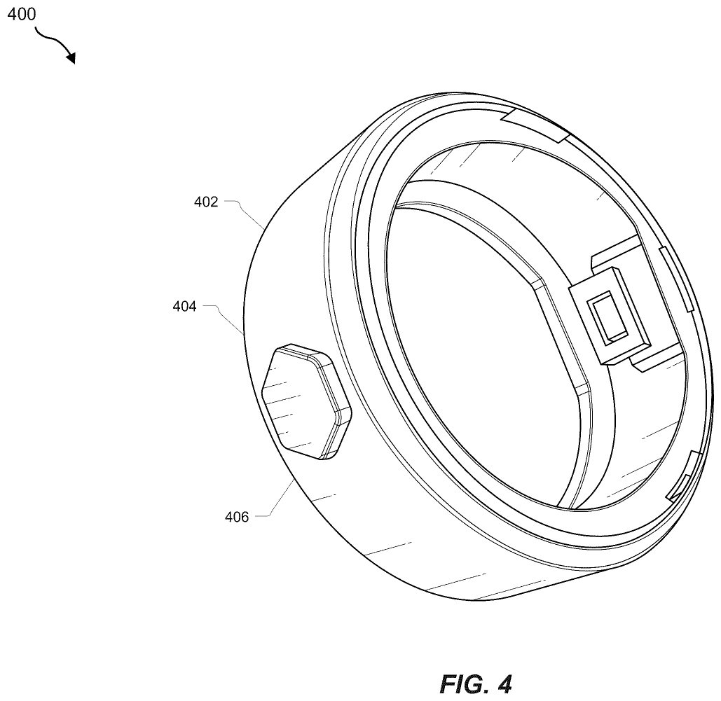

As described above in connection with FIG. 1, a wearable device such as wearable 150 may include a device designed and/or intended to be worn by a wearer and/or integrated into clothing, such as a smart ring, a smart band, a smart watch, a smartphone, and so forth. FIG. 4. shows a perspective view 400 of an example smart ring device 402 that may be used in connection with some embodiments of the systems and methods disclosed herein. Smart ring device 402 may include an NFC antenna 404 and a secured storage device 406. In some examples, NFC antenna 404 may implement at least part of a wireless connection such as wireless connection 216 and/or secured storage device 406 may implement a secured storage device like secure storage device 152.

In some examples, a request such as request 204 may include and/or refer to a communication or instruction initiated by the wearer 206 that may indicate an intent to acquire or purchase a particular item, such as item 208. This request can be initiated in various ways, such as by the wearer physically presenting the item at the point-of-sale device or scanning it, or by inputting the item's details in an online shopping interface, among other suitable methods.

In at least some examples, the request may include or be associated with an identifier such as identifier 224. The identifier may uniquely identify the specific item the wearer intends to acquire. This identifier may include, without limitation, a barcode, a QR code, or any other form of identifier associated with the item and that may enable one or more of the systems disclosed herein to retrieve detailed information about the item, such as its ingredients, for subsequent operations such as allergen checks.

As also illustrated in FIG. 3 at step 310, one or more of the systems described herein may receive, from a secure storage device included in the wearable that securely maintains a set of restrictions associated with the wearer, the set of restrictions associated with the wearer. For example, receiving module 104 may, as part of computing device 202 in FIG. 2, cause computing device 202 to receive, from secure storage device 152 included in wearable 150, wearer restriction data 154.

Receiving module 104 may receive request 204 from wearer 206 and wearer restriction data 154 in a variety of contexts. For example, as indicated in FIG. 2, wearable 150, computing device 202, and, in some examples, point-of-sale device 218 may be in communication via wireless connection 216. Additionally or alternatively, in some examples, wearable 150, computing device 202, and point-of-sale device 218 may be in communication via network 226. Hence, in some examples, receiving module 104 may receive request 204 from wearer 206 via point-of-sale device 218 and/or via wearable 150.

By way of illustration, wearer 206 may use reader 220 included in point-of-sale device 218 to scan identifier 224 associated with item 208. This may manifest a request by wearer 206 to acquire (e.g., purchase) item 208, and point-of-sale device 218 may communicate this request, via network 226, to receiving module 104, which may receive the request (e.g., request 204). Likewise, wearer 206 may attempt to use a wireless payment functionality (e.g., NFC) of wearable 150 to pay for item 208. Wearer 206 may thus interact with a suitable wireless payment feature of point-of-sale device 218 which may cause point-of-sale device 218 to contemporaneously receive, from secure storage device 152 included in wearable 150, wearer restriction data 154. Point-of-sale device 218 may transmit wearer restriction data 154 to receiving module 104, which may thus receive wearer restriction data 154.

Returning to FIG. 3, at step 320, one or more of the systems described herein may identify at least one potential restriction associated with the item. For example, identifying module 106 may, as part of computing device 202 in FIG. 2, cause computing device 202 to identify potential restriction 210 associated with item 208.

A potential restriction such as potential restriction 210 may include any limitation or constraint associated with the item (e.g., item 208) that may pose a risk or cause harm to the wearer of the wearable device (e.g., wearer 206). Such restrictions could be due to the presence of allergens, substances causing intolerance, ingredients violating the wearer's dietary preferences or lifestyle choices, or any other factor that could impact the wearer's health, legal status, personal preferences, and so forth. For instance, if item 208 is a food product, potential restriction 210 could be the presence of a common allergen such as peanuts. Identifying module 106 may identify one or more potential restrictions to compare the identified potential restrictions against the wearer's personalized restriction data (e.g., wearer restriction data 154), enabling the embodiments of the systems and methods disclosed herein to determine if the item is safe or suitable for the wearer to acquire.

Identifying module 106 may identify potential restriction 210 in a variety of contexts. For example, identifying module 106 may access potential restriction data 142 in data store 140, which may include, as noted above, a structured data set of attributes (e.g., ingredients, aspects, ownership identifiers, etc.) associated with items. By analyzing potential restriction data 142, identifying module 106 may identify potential restriction 210. In some examples, potential restriction data 142 may include and/or be organized in accordance with identifiers associated with items, such as identifier 224. Hence, in examples where request 204 includes identifier 224, identifying module 106 may use identifier 224 to identify one or more potential restrictions (e.g., ingredients included in) item 208.

Returning to FIG. 3, at step 330, one or more of the systems described herein may determine, based on the set of restrictions associated with the wearer and the at least one potential restriction, a restriction associated with the item and the wearer. For example, determining module 108 may, as part of computing device 202 in FIG. 2, cause computing device 202 to determine, based on wearer restriction data 154 and potential restriction 210, a shared restriction 212.

Shared restriction 212 may include or represent a constraint that is common to both the wearer of the wearable device (as denoted by wearer restriction data 154) and the potential restrictions (like potential restriction 210) associated with the item in question.

Determining module 108 may determine shared restriction 212 in a variety of contexts. For example, determining module 108 may assess the wearer's restrictions (such as health conditions, allergies, or lifestyle preferences) in conjunction with the potential restrictions of the item the wearer is attempting to acquire. If there is an overlap—a restriction that applies both to the wearer and the item—this determining module 108 may identify and/or select the overlapping restriction as shared restriction 212. For instance, if the wearer has a peanut allergy (a restriction in wearer restriction data 154) and the item contains peanuts (a potential restriction 210), determining module 108 may determine that the common factor—the presence of peanuts—is a shared restriction, and may identify and/or select the shared restriction as shared restriction 212. This information may then be used to help ensure the safety and/or suitability of the wearer's intended acquisitions and/or purchases.

Returning to FIG. 3, at step 340, one or more of the systems described herein may execute at least one warning action in response to determining the restriction associated with the item and the wearer. For example, executing module 110 may, as part of computing device 202 in FIG. 2, cause computing device 202 to execute warning action 214 in response to determining shared restriction 212.

In some examples, a warning action like warning action 214 may include and/or refer to a responsive measure that one or more of modules 102 (e.g., executing module 110) may execute when one or more of modules 102 determines that a shared restriction (e.g., shared restriction 212) exists between the wearer's restrictions and the potential restrictions of an item.

In some examples, a warning action may include sending a notification or alert to the wearer of the wearable device, the point-of-sale device, and/or another party, advising them about the shared restriction. Additionally or alternatively, the warning action may include, without limitation, displaying a message, emitting an audio signal, vibrating the wearable device, halting a transaction, and so forth.

The warning action may alert the wearer (or, in some examples, an operator of the point-of-sale device) to a potential risk or harm associated with the item. This may further provide an opportunity to prevent a potentially dangerous or unsuitable purchase or acquisition of an item. For example, if the shared restriction 212 is a peanut allergy and item 208 contains peanuts, warning action 214 may include issuing an alert via an output device (e.g., via user interface 222 and/or a notification functionality of wearable 150) to prevent the wearer from unknowingly purchasing and consuming a product that contains allergens.

In some examples, the warning action may include preventing of the transaction. In additional or alternative examples, the warning action may include pausing of the transaction, presenting an approval control via a user interface, and resuming the transaction upon receiving an approval via the approval control. In some examples, the approval must be provided by the wearer, thus indicating that the wearer is aware of any potential risk and/or accepts the potential risk.

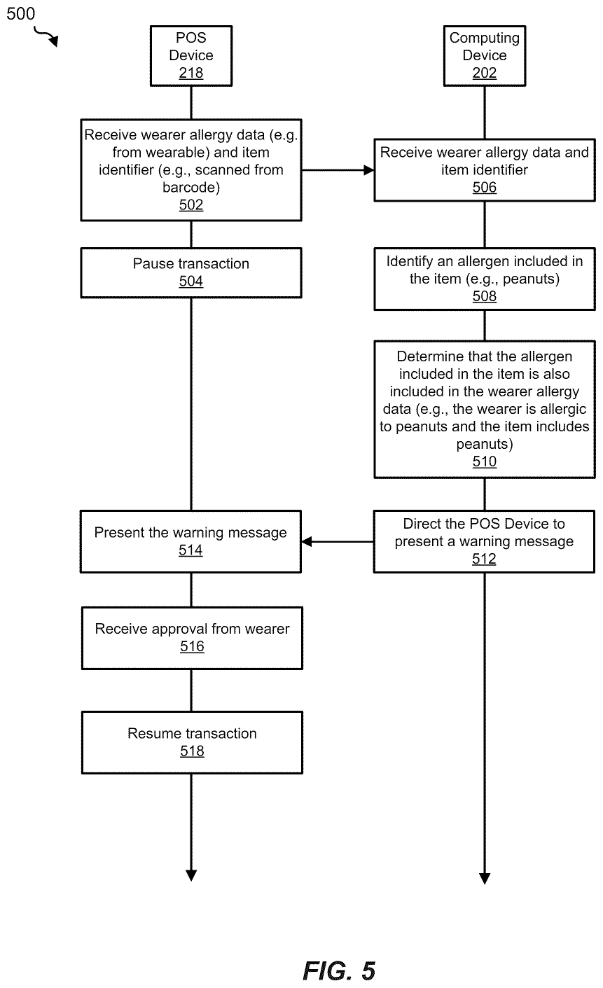

By way of example, FIG. 5 includes a sequence diagram 500 that illustrates operations that may be executed by point-of-sale device 218 and computing device 202 during an example transaction. Note that this example is provided to illustrate possible operations and is not intended to limit the scope of the claims. In this example, wearer restriction data 154 indicates that wearer 206 has an allergy to peanuts and wearer 206 is attempting to purchase a food item (e.g., item 208) that contains peanuts.

At operation 502, point-of-sale device 218 receives wearer allergy data (e.g., wearer restriction data 154) and an identifier associated with the item wearer 206 is attempting to purchase (e.g., a code or identifier encoded by a barcode or QR code, such as identifier 224). Point-of-sale device 218 transmits this data to computing device 202. At operation 504, point-of-sale device 218 pauses the transaction (e.g., a transaction involving conveyance of an item to wearer 206) pending an outcome of the following operations.

At operation 506, computing device 202 receives the wearer allergy data and the identifier (e.g., wearer restriction data 154 and identifier 224). At operation 508, computing device 202 identifies (e.g., by accessing potential restriction data 142) an allergen included in the item (e.g., peanuts). At operation 510, computing device 202 determines that the allergen included in the item is also included in the wearer's allergy data (e.g., wearer restriction data 154 indicates that wearer 206 has an allergy to peanuts and potential restriction data 142 indicates that the item includes peanuts).

At operation 512, computing device 202 executes a warning by directing point-of-sale device 218 to present a warning message via a user interface (e.g., warning action 214 via user interface 222). At operation 514, point-of-sale device 218 presents the warning message. At operation 516, point-of-sale device 218 receives (e.g., via user interface 222) approval from the wearer to continue the transaction. At operation 518, point-of-sale device 218 resumes the transaction.

It may be important to ensure that wearer restriction data 154 is always up-to-date. Hence, in some examples, one or more of modules 102 may also receive an additional potential restriction and may transmit the additional potential restriction to the wearable for inclusion in the set of restrictions associated with the wearer. In some examples, an additional potential restriction may include a revised set of potential restrictions and/or an instruction to remove one or more potential restrictions from wearer restriction data 154.

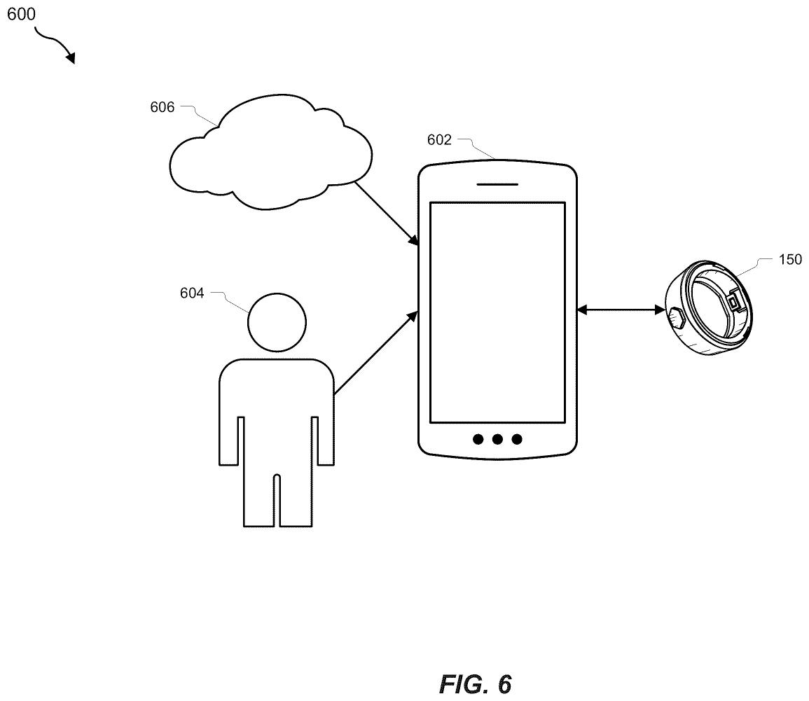

FIG. 6 includes a block diagram 600 that illustrates receiving an additional potential restriction and transmitting the additional potential restriction to the wearable. As shown, FIG. 5 includes wearable 150 in communication with a user data input device 602. User data input device 602 may include any suitable device or instrument that may facilitate reception of additional potential restrictions and transmission of the additional potential restrictions to wearable 150 and/or secure storage device 152 included in wearable 150. As shown, user data input device 602 may include a smartphone. In additional or alternative examples, user data input device 602 may include, without limitation, a personal computing device, a web-based application, a server, and so forth.

FIG. 6 also includes a user 604 and a remote data management system 606. User 604 may include any person (e.g., wearer 206, a parent or guardian of wearer 206, a government or law enforcement officer, etc.) who may interact with user data input device 602 to provide additional potential restrictions for inclusion in wearer restriction data 154. Likewise, remote data management system 606 may include any automated or remotely operated data management system that may provide one or more additional potential restrictions. For example, and without limitation, remote data management system 606 may include a healthcare provider cloud-based data management system, an employer-operated data management system, a governmental or law enforcement-operated data management system, and so forth.

Hence, in some examples, one or more of modules 102 may receive an additional potential restriction by receiving the additional potential restriction from a user data input device (e.g., user data input device 602) and/or from a remote data management system (e.g., remote data management system 606). Moreover, some examples, as shown in FIG. 6, the remote data management system may provide the additional potential restriction via the user data input device.

As may be clear from the foregoing, embodiments of the systems and methods disclosed herein may provide many benefits over conventional or traditional options. The present disclosure describes a sophisticated system that interacts with wearable devices, point-of-sale devices, and a computing device to protect users from potential risks related to the purchase of certain items. In simple terms, when a user, wearing a device equipped with personalized restriction data, wants to buy an item, the system checks if the item poses any risk to the user. This check is done based on potential restrictions associated with the item, such as allergens in a food product. If a shared restriction (a risk common to both the user's restrictions and the item's potential restrictions) is identified, the system can take warning actions such as presenting an alert and/or pausing or stopping the transaction to avoid harm to the user.

Traditional transaction systems usually rely on the user's knowledge and diligence to avoid purchasing items that may pose risks. Embodiments of the present disclosure, however, may proactively identify potential hazards based on personalized restriction data and potential restrictions of the item, reducing the reliance on a user's memory or awareness. Furthermore, embodiments of the systems and methods disclosed herein may intervene in real-time at the point of sale, providing immediate warnings and even halting transactions if necessary. This offers superior risk prevention and user protection compared to conventional approaches, particularly in (though not limited to) scenarios involving allergens, dietary preferences, or lifestyle choices.

As detailed above, the computing devices and systems described and/or illustrated herein broadly represent any type or form of computing device or system capable of executing computer-readable instructions, such as those contained within the modules described herein. In their most basic configuration, these computing device(s) may each include at least one memory device and at least one physical processor.

Although illustrated as separate elements, the modules described and/or illustrated herein may represent portions of a single module or application. In addition, in certain embodiments one or more of these modules may represent one or more software applications or programs that, when executed by a computing device, may cause the computing device to perform one or more tasks. For example, one or more of the modules described and/or illustrated herein may represent modules stored and configured to run on one or more of the computing devices or systems described and/or illustrated herein. One or more of these modules may also represent all or portions of one or more special-purpose computers configured to perform one or more tasks.

In addition, one or more of the modules described herein may transform data, physical devices, and/or representations of physical devices from one form to another. For example, one or more of the modules recited herein may receive restriction data to be transformed, transform the restriction data, output a result of the transformation to identify a shared restriction, use the result of the transformation to execute a warning action, and store the result of the transformation to track a history of warning actions and/or attempts to purchase items with one or more restrictions. Additionally or alternatively, one or more of the modules recited herein may transform a processor, volatile memory, non-volatile memory, and/or any other portion of a physical computing device from one form to another by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media include, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems. In some examples, non-transitory-type media may be referred to as a non-transitory computer-readable medium.

The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed. The various exemplary methods described and/or illustrated herein may also omit one or more of the steps described or illustrated herein or include additional steps in addition to those disclosed.

The preceding description has been provided to enable others skilled in the art to best utilize various aspects of the exemplary embodiments disclosed herein. This exemplary description is not intended to be exhaustive or to be limited to any precise form disclosed. Many modifications and variations are possible without departing from the spirit and scope of the instant disclosure. The embodiments disclosed herein should be considered in all respects illustrative and not restrictive. Reference should be made to the appended claims and their equivalents in determining the scope of the instant disclosure.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of” Finally, for ease of use, the terms “including” and “having” (and their derivatives), as used in the specification and claims, are interchangeable with and have the same meaning as the word “comprising.”

What is claimed is:

1. A computer-implemented method comprising:
receiving:
a request from a wearer of a wearable for the wearer to acquire an item;
from a secure storage device included in the wearable that securely maintains a set of restrictions associated with the wearer, the set of restrictions associated with the wearer;
identifying at least one potential restriction associated with the item;
determining, based on the set of restrictions associated with the wearer and the at least one potential restriction, a restriction associated with the item and the wearer; and
executing at least one warning action in response to determining the restriction associated with the item and the wearer, the warning action enabling functionality for controlling actions by the wearable.

2. The computer-implemented method of claim 1, wherein:
the set of restrictions associated with the wearer comprises allergy data associated with the wearer;
the at least one potential restriction associated with the item comprises a potential allergen associated with the item; and
determining, based on the set of restrictions associated with the wearer and the at least one potential restriction, the restriction associated with the item and the wearer comprises determining, based on the allergy data associated with the wearer and the potential allergen, a potential allergy risk of the item to the wearer.

3. The computer-implemented method of claim 1, wherein receiving the set of restrictions associated with the wearer comprises receiving the set of restrictions associated with the wearer via a wireless communication protocol.

4. The computer-implemented method of claim 3, wherein the wireless communication protocol comprises at least one of:
a near-field communication (NFC) wireless communication protocol; and
a Bluetooth wireless communication protocol.

5. The computer-implemented method of claim 1, wherein receiving the request from the wearer of the wearable for the wearer to receive the item and receiving the set of restrictions associated with the wearer comprises receiving the request from the wearer of the wearable to receive the item and receiving the set of restrictions associated with the wearer at a point-of-sale during a purchase by the wearer of the item.

6. The computer-implemented method of claim 1, wherein:
the computer-implemented method further comprises maintaining, within a structured data set of attributes, a set of attributes associated with the item;
identifying the at least one potential restriction associated with the item comprises:
analyzing an identifier associated with the item;
identifying, from the structured data set of attributes, the set of attributes associated with the item based on the identifier associated with the item;
comparing the set of attributes associated with the item and the set of restrictions associated with the wearer; and
determining that the at least one potential restriction is included in both the set of attributes associated with the item and the set of restrictions associated with the wearer.

7. The computer-implemented method of claim 1, further comprising:
receiving at least one additional potential restriction; and
transmitting the at least one additional potential restriction to the wearable for inclusion in the set of restrictions associated with the wearer.

8. The computer-implemented method of claim 7, wherein receiving the at least one additional potential restriction comprises receiving the at least one additional potential restriction from a remote data management system.

9. The computer-implemented method of claim 7, wherein receiving the at least one additional potential restriction comprises receiving the at least one potential restriction via a user data input device.

10. The computer-implemented method of claim 1, wherein executing the at least one warning action comprises presenting a warning message via an output device.

11. The computer-implemented method of claim 1, wherein executing the at least one warning action in response to determining the restriction associated with the item and the wearer comprises:
pausing a transaction involving conveyance of the item to the wearer;
presenting an approval control via a user interface; and
upon receiving an approval via the approval control, resuming the transaction involving conveyance of the item to the wearer.

12. The computer-implemented method of claim 11, wherein:
presenting the approval control via the user interface comprises presenting a wearer approval control via the user interface; and
receiving the approval via the wearer approval control comprises receiving a wearer approval via the wearer approval control.

13. A system comprising:
a receiving module, stored in memory, that receives:
a request from a wearer of a wearable for the wearer to acquire an item;
from a secure storage device included in the wearable that securely maintains a set of restrictions associated with the wearer, the set of restrictions associated with the wearer;
an identifying module, stored in memory, that identifies at least one potential restriction associated with the item;
a determining module, stored in memory, that determines, based on the set of restrictions associated with the wearer and the at least one potential restriction, a restriction associated with the item and the wearer; and
an executing module, stored in memory, that executes at least one warning action in response to determining the restriction associated with the item and the wearer, the warning action enabling functionality for controlling actions by the wearable.

14. The system of claim 13, wherein the receiving module receives the set of restrictions associated with the wearer by receiving the set of restrictions associated with the wearer via a wireless communication protocol, the wireless communication protocol comprising at least one of:
a near-field communication (NFC) wireless communication protocol; and
a Bluetooth wireless communication protocol.

15. The system of claim 13, wherein the receiving module receives the request from the wearer of the wearable for the wearer to receive the item and receives the set of restrictions associated with the wearer by receiving the request from the wearer of the wearable to receive the item and receiving the set of restrictions associated with the wearer at a point-of-sale during a purchase by the wearer of the item.

16. The system of claim 13, wherein:
the identifying module further maintains, within a structured data set of attributes, a set of attributes associated with the item;
the identifying module identifies the at least one potential restriction associated with the item by:
analyzing an identifier associated with the item;
identifying, from a structured data set of attributes, the set of attributes associated with the item based on the identifier associated with the item;
comparing the set of attributes associated with the item and the set of restrictions associated with the wearer; and
determining that the at least one potential restriction is included in both the set of attributes associated with the item and the set of restrictions associated with the wearer.

17. The system of claim 13, wherein the executing module executes the at least one warning action by presenting a warning message via an output device.

18. The system of claim 13, wherein the executing module executes the at least one warning action in response to determining the restriction associated with the item and the wearer by:
pausing a transaction involving conveyance of the item to the wearer;
presenting an approval control via a user interface; and
upon receiving an approval via the approval control, resuming the transaction involving conveyance of the item to the wearer.

19. A non-transitory computer-readable medium comprising computer-readable instructions that, when executed by at least one processor of a computing system, cause the computing system to:
receive:
a request from a wearer of a wearable for the wearer to acquire an item;
from a secure storage device included in the wearable that securely maintains a set of restrictions associated with the wearer, the set of restrictions associated with the wearer;
identify at least one potential restriction associated with the item;
determine, based on the set of restrictions associated with the wearer and the at least one potential restriction, a restriction associated with the item and the wearer; and
execute at least one warning action in response to determining the restriction associated with the item and the wearer, the warning action enabling functionality for controlling actions by the wearable.

20. The non-transitory computer-readable medium of claim 19, wherein the computer-readable instructions, when executed by the at least one processor of the computing system, further cause the computing system to execute the at least one warning action in response to determining the restriction associated with the item and the wearer by:
pausing a transaction involving conveyance of the item to the wearer;
presenting an approval control via a user interface; and
upon receiving an approval via the approval control, resuming the transaction involving conveyance of the item to the wearer.
